(19) United States
(12) Patent Application Publication
(10) Pub. No.: US 2010/0152599 A1
(43) Pub. Date: Jun. 17, 2010

(54) ORAL APPLIANCE COMPLIANCE MONITORING SYSTEM

(76) Inventors: James Brian DuHamel, Valley Springs, CA (US); Christopher Hause, Valley Springs, CA (US); Deepak Shrivastava, Stockton, CA (US)

Correspondence Address: BEESON SKINNER BEVERLY LLP, ONE KAISER PLAZA, SUITE 750, OAKLAND, CA, 94612, US

(21) Appl. No.: 12/560,394

(22) Filed: Sep. 15, 2009

Related U.S. Application Data
(60) Provisional application No. 61/097,113, filed on Sep. 15, 2008.
(60) Provisional application No. 61/099,557, filed on Sep. 23, 2008.
(60) Provisional application No. 61/184,651, filed on Jun. 5, 2009.

(51) Int. Cl. A61B 5/08; H04L 29/06

(52) U.S. Cl. 600/532; 713/153

(57) ABSTRACT

An oral appliance compliance monitoring system and method comprises an oral appliance suitable for wearing in a patient's oral cavity during sleeping periods, the oral appliance having one or more sensors measuring a variety of conditions such as oxygen saturation levels in the oral cavity mucosa. The data generated by the sensor is continuously transmitted to a local scanner which is in communication with a central computer. The computer interprets the data to determine if the patent is wearing the oral appliance in compliance with a prescribed treatment regimen for breathing-related sleep disorders. Remotely located computers are authorized to receive the streamed data to enable remote monitoring of compliance in real time by a plurality of patients with treatment regimens.

### FIELD OF INVENTION

The present invention relates generally to monitoring the use of oral appliances being used for treatment of breathing-related sleep disorders to determine compliance with treatment regimens.

### BACKGROUND OF THE INVENTION

A sizable percentage of patients diagnosed with breathing-related sleep disorders are prescribed and use an oral appliance. There is, however, no viable method for verifying compliance with the use of the prescribed oral appliance.

Oral Appliance Therapy was accepted by the American Academy of Sleep Medicine in February of 2006. Existing art oral appliance compliance systems are able to monitor when the oral appliance is worn but are limited to displaying the information on the computer only after scanning of a chip in the device is completed. This information is displayed in a chronological graph that can be printed out for a patient's records. No technology exists that makes the monitoring information instantaneously readable by interested parties from remote locations. The present invention is able to fulfill such needs.

No prior art devices embed sensors into oral appliances for monitoring oxygen saturation, blood pressure, snoring levels, breathing patterns, body positions, brain wave activity, muscle activity, tongue position and intraoral pressure. Rather, these activities are presently monitored by attaching sensors to other parts of the body and relaying information to computers via wires and recently wirelessly. For example, compliance monitoring systems representing the current standard of care record information through chips in continuous positive airway pressure (CPAP) machines. Air flow patterns and air pressure are used to monitor compliance of the CPAP system. Oxygen levels, heart rate, body position, etc. are not monitored.

The prior solutions do not completely solve this problem because information scanned into a computer is only available at the physical location of the computer. No algorithms have been previously developed to monitor snoring or sleep patterns using temperature and vibration centers.

Moreover, no prior art systems or devices have placed electronic chips into oral appliances because, given that the focus in the industry in the area of orthodontics, there was no intention to monitor or treat sleep disordered breathing with oral appliance therapy.

The invention described herein enables objectively documenting that patients are wearing oral appliances during periods of sleep and makes this information available as required on a scheduled basis to interested parties and without the patient having to go to the treatment facility. The invention demonstrates that an oral appliance can be used to correct a patient's sleep disorder breathing.

The invention addresses the need for a compliance system and enables the following functions and assessments:
(1) Compliance Monitoring Component
a. Objective documentation that patients suffering from breathing-related sleep disorders are compliant with prescribed regimens for wearing an oral appliance on a nightly basis or whenever asleep.
b. Collection, processing and relaying the compliance-related information immediately to interested parties such as physicians, employers, and insurance companies.
(2) Diagnostic System Component
a. Objective documentation of the presence of sleep disordered breathing using a portable device in the form of an oral appliance.
b. Utilizing pressure changes inside the oral cavity to document the reduction in or absence of air flow throughout the sleep period.
c. Utilizing measurable oxygen saturation levels in the oral cavity mucosa during the sleep period to determine breathing activity.
d. Collection, processing and relaying the diagnosis related information immediately to the interested parties (physician, employer, insurance companies and others).
(3) Therapeutic System Component
a. Verifying that the oral appliance is successful in correcting the sleep disordered breathing when worn during the sleep period.
b. Relaying physiological information pertaining to the patient's health including, but not limited to, resolution of snoring, cessation of breathing (apnea), limitation in airflow (hypopnea) or oxygen desaturation.
c. Collection, processing and relaying the therapy related information immediately to the interested parties (physician, employer, insurance companies and others).

### BRIEF DESCRIPTION OF THE DRAWINGS

FIG. 1 is a representative view of an oral appliance showing a sensor embedded in the sublingual area.

FIG. 2 is a representative diagram of a pulse oximetry sensor.

FIG. 3 is a representative diagram of a pressure transducer.

FIG. 4 is a schematic diagram of an oral appliance compliance monitoring system according to the invention.

### DETAILED DESCRIPTION OF THE ILLUSTRATED EMBODIMENT

The invention is the result of the development of a software program and electronic network that facilitates data collection in a patient's natural environment. Such data can be remotely uploaded to the hosted web-site via an on-site scanner and a central computer that is readily accessible to the interested parties.

An oral appliance 101 may be worn on upper or lower arches and may be made of acrylics, resin, laminates or any other material approved by the FDA for oral use. See FIG. 1. Sensors may reflect off the teeth or soft tissue.

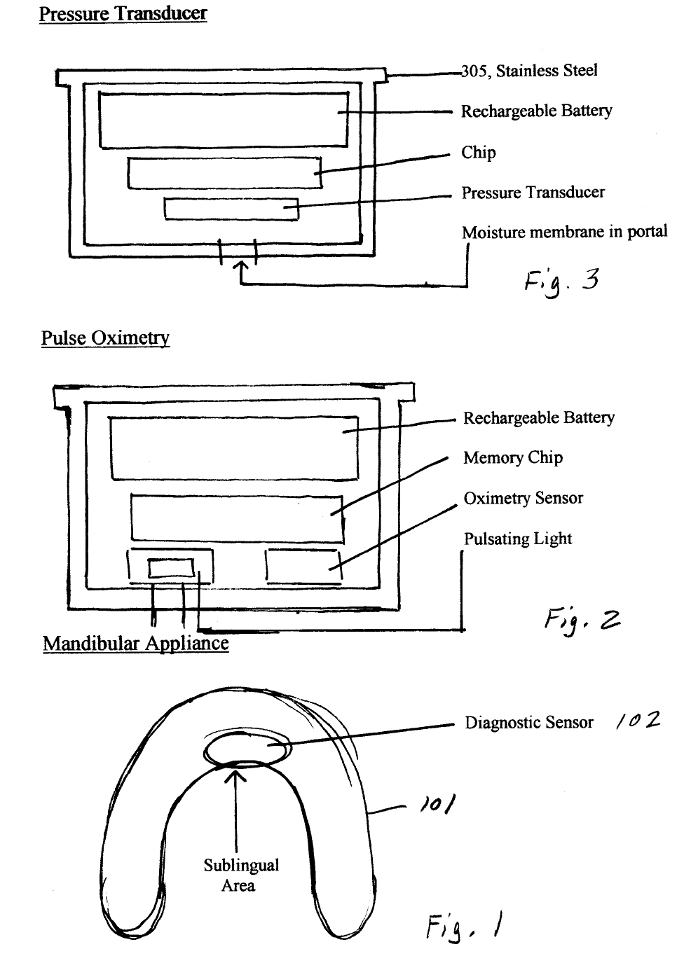

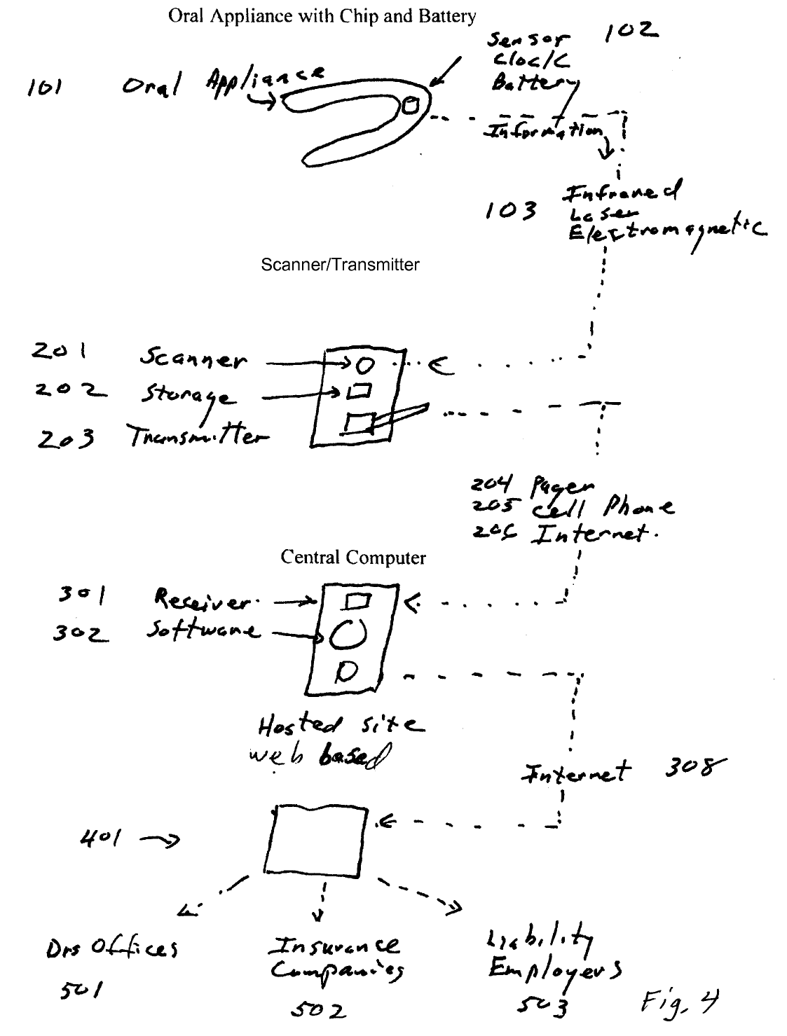

A variety of parameters can be monitored by incorporating different interfaces into the oral appliance. Sensor chips 102 may be altered depending on the parameter being monitored, including but not limited to light, temperature, vibration, sound, oximetery, EMG, EEG, pulse, position, noise, Ph, atmospheric pressure, or tactile pressure. A pulse oximetry sensor, such as that illustrated in FIG. 2, will measure oxygen levels. A pressure transducer, such as that illustrated in FIG. 3, senses pressure differentials. Sensors 102 record time and date of events and have a power supply attached, e.g., a battery. See FIGS. 2 and 3. The battery may be replaceable or be rechargeable by the scanner unit. Each chip will be identified by a serial number that will be recognized by the scanner. With additional reference to FIG. 4, the information from the chip will be transmitted at 103 to the scanner via infrared electromagnetic waves or blue tooth technology.

The scanner receives the data at 102 and stores the information on a second chip when the appliance is docked on the scanner receiver.

The information will be stored and accumulated at 202. The information can be transmitted by command at the scanner or pulled by command off the central computer. The information in the scanner will erase by command at the scanner or instructions from the central computer.

The information will be encrypted and transferred to the central computer at 203 from the scanner by various modalities including, but not limited to, pagers, phone lines, the internet, cell phones, and radio waves.

The central computer will receive the data in an encrypted form at 301.

Software program will analyze the data at 302 via an algorithm for different applications including:

a. Compliance of use as prescribed;

b. Measurement of physiological parameters including temperature, oxygen saturation level, heart rate, snoring level, air flow, intraoral pressure, tactile pressure, muscle activity and bruxism; and

c. Analysis of medical conditions such as: sleep disordered breathing, snoring, heart disease, respiratory dysfunction, swallow disorders, temporo-mandibular joint (TMJ) disorder, parafunctional activities.

The software customizes and presents the data at 303 according to user requirements and preference, such as, hours of wearing the appliance. Parameters of measurement like sampling rate and display frequency can be set by the monitoring personnel.

The invention provides for exception reporting. Noncompliant patients will be flagged by the computer software and proactive actions to assist the patient will be recommended.

The central computer will download information at 304 to a hosted website that will be accessible to authorized personnel through access codes.

The computer will automatically charge or bill users at 305 for input from sensors and information retrieved from the computer or website.

The computer will notify liable and at risk parties at 306 of noncompliance or changes that will affect the patient's health or safety. Liable parties may include employers, patients, physicians, dentists, or insurance companies.

The hosted website will be given the appropriate information at 308 by encrypted media via the internet or secure electronic mail.

The hosted web page will be accessible at 401 by authorized personnel that have supporting software. Participating medical offices will have all medical and compliance related information available online.

Employers will have pre-authorized compliance status information. Only insurance companies will have pre-authorized compliance status information.

The invention has the advantages over the prior art that it has remote data collection and transmittal capability, provides display of physiologic data, and has diagnostic and therapeutic capabilities incorporated within the oral appliance. No other monitoring systems use electronic smart chip in oral appliances.

Prior art systems provided no solution to compliance monitoring by using sensors and chips to verify and document compliance. Implementation of the system eliminates the need for patients going'to the dental professional's office for appointments, eliminates the need for printing out graphs and faxing or emailing information to interested parties, provides an integrated system for disseminating information to interested persons. Compared to ambulatory sleep studies, an oral appliance is more comfortable, better tolerated by patients, reduces the need for office appointments, and records information continuously over many sleep periods.

Finally, this invention will convert oral appliance therapy, a well established therapeutic modality in to a convenient diagnostic system that can monitor various parameters in patient's habitual environment over a period of many nights precluding the effects of sleeping in the sleep laboratory known as “first night effect,” night to night variability in sleep architecture and influence of substance use like social alcohol consumption. The clinical information yield of our invention in above mentioned situations is likely to be very high compared to laboratory based full polysomnography.

There have thus been described and illustrated certain preferred embodiments of an oral applicant compliance monitoring system and method according to the invention. Although the present invention has been described and illustrated in detail, it is clearly understood that the same is by way of illustration and example only and is not to be taken by way of limitation, the spirit and scope of the present invention being limited only by the terms of the appended claims and their legal equivalents.

We claim:

1. An oral appliance compliance monitoring system comprising:
an oral appliance having an electronic sensor, a transmitter in communication with said sensor, and a power source for energizing said sensor and said transmitter, said sensor capable of continuously monitoring oxygen saturation levels in oral cavity mucosa,
a central computer in electronic communication with said transmitter, said computer having a memory and a processor, said memory for storing data generated by said sensor related to said oxygen saturation levels and received from said transmitter, said processor for comparing measured oxygen saturation levels against predetermined oxygen saturation levels associated with the presence or absence of said oral appliance in an oral cavity to verify presence of said oral appliance in the oral cavity.

2. The oral appliance compliance monitoring system of claim 1 further comprising:
said transmitter capable of continuously transmitting data received from said sensor,
one or more authorized computers in communication with said central computer, each said authorized computer capable of receiving encrypted data from said central computer,
said central computer capable of receiving said data from said sensor, encrypting said data, and sending said encrypted data to said authorized computers.
